(19) United States
(12) Patent Application Publication
(10) Pub. No.: US 2003/0060808 A1
(43) Pub. Date: Mar. 27, 2003

(54) Telemedical method and system

(76) Inventor: Peter J. Wilk, New York, NY (US)

Correspondence Address: COLEMAN SUDOL SAPONE, P.C., 714 COLORADO AVENUE, BRIDGEPORT, CT, 06605-1601, US

(21) Appl. No.: 09/790,308

(22) Filed: Feb. 22, 2001

Related U.S. Application Data
(63) Continuation-in-part of application No. 09/678,722, filed on Oct. 4, 2000.

(51) Int. Cl. A61B 17/00

(52) U.S. Cl. 606/1; 128/903

(57) ABSTRACT

Using a mobile medical facility such as a surgical operating room, a physician may perform various diagnostic and surgical instruments on a patient located at a distance from the physician. The mobile medical facility is carried on a vehicle such as a truck, a ship or and aircraft. Generally, it is contemplated that the vehicle is stationary during the performance of a medical procedure. However, it is possible to conduct an examination or treatment even when the medical facility is in motion. Control signals and feedback signals are transmitted redundantly, over multiple communications pathways.

### BACKGROUND OF THE INVENTION

This invention relates to a medical system. This invention also relates to an associated medical method. More specifically, this invention relates to a telemedical system and an associated telemedical method. The system and the method of the present invention are particularly useful in the various areas of endoscopic surgery, including flexible endoscopic surgery, laparoscopic surgery, thoracoscopic surgery, arthroscopic surgery, etc.

Telemedicine refers to the practice of medicine at a distance. The patient and the physician are not located at the same place during the gathering of information from the patient or during the performance of a treatment on the patient. Telemedicine is considered of particular advantage in situations where a sick or injured individual is in a practically inaccessible location. Such situations arise in circumstances of exploration or military conflict or simply where the individual is located at a distance from centers of civilization.

U.S. Pat. Nos. 5,217,003, 5,217,453 and 5,368,015 disclose a telesurgical system wherein a surgeon is situated at a such a distance from a patient that direct manual control of surgical instrumentation is impossible. The surgeon and patient may, for instance, be situated in different cities. A surgical operation is implemented via a telecommunications link and robotics at the location of the patient. The surgeon manipulates controllers in response to real-time input regarding the condition of the patient. Signals generated by the controllers in response to manipulations by the surgeon are transmitted over the telecommunications link to a control computer at the patient's location. That computer operates the robotics in response to the surgeon generated instructions.

This feedback provided to the remote surgeon includes images captured with a camera or other scanner. Other parameters regarding the condition of patient P, such as temperature, heart rate, oxygen consumption, brain wave activity, and blood sugar level, may also be automatically sensed, encoded and transmitted to a remote computer at the surgeon's location for providing the surgeon in real time with all information necessary for performing the surgery successfully.

As described in U.S. Pat. Nos. 5,217,003, 5,217,453 and 5,368,015, a camera may be provided on an endoscopic instrument which is inserted into the patient for viewing internal organs of the patient in a minimally invasive procedure. Such minimally invasive endoscopic procedures include laparoscopic, arthroscopic, thoracoscopic and flexible endoscopic procedures.

### OBJECTS OF THE INVENTION

An object of the present invention is to provide an improved method and/or apparatus for performing remotely executed medical procedures including, but not limited to, surgical operations. In such remotely executed medical procedures, one or more medical personnel (e.g., physicians) are beyond a range of direct manual contact with the patient.

Another object of the present invention is to provide such a method and/or apparatus wherein the control exercisable by the operating physician is enhanced.

It is a further object of the present invention to provide such a method and/or apparatus which is particularly usable with endoscopic and/or laparoscopic equipment.

Another, more particular, object of the present invention is to provide a method and apparatus which facilitates the performance of operations by surgeons from all over the world.

These and other objects of the present invention will be apparent from the drawings and descriptions herein. It is to be noted that each object is attainable by one or more embodiments of the present invention. However, not every embodiment necessarily meets every object set forth herein.

### SUMMARY OF THE INVENTION

The present invention is directed to a mobile medical facility such as a surgical operating room. The invention contemplates moving the medical facility to the location of the patient. Various diagnostic and surgical instruments in the mobile medical facility are operated under remote control so that a physician at a distant location is capable of examining and treating the patient.

Pursuant to the present invention, it is preferred that control signals transmitted from the physician to the movable medical facility containing the patient are transmitted redundantly along at least two separate transmission pathways. In addition, information as to the patient's condition is transmitted in real-time, continuously, redundantly over at least two transmission pathways to the remote physician.

The mobile medical facility is carried on a vehicle such as a truck, a ship or and aircraft. Generally, it is contemplated that the vehicle is stationary during the performance of a medical procedure. However, it is possible to conduct an examination or treatment even when the medical facility is in motion.

The present invention facilitates the provision of medical care particularly where patients are in practically inaccessible locations or where there is a dearth of qualified practitioners to adequately service the relevant populace. The system enables an improved matching of medical care to patients. Physician specialists are capable of treating more patients scattered over a wider area, in fact, over the entire world and beyond.

A medical system comprises more specifically, in accordance with the present invention, a self-propelled vehicle having a cargo compartment, a sensor device disposed in the cargo compartment for collecting information on a patient in the cargo compartment, at least one control device disposed in the cargo compartment for operating a medical instrument relative to the patient, and communications components including a signal receiver and a transmitter disposed on the vehicle. The transmitter is operatively connected to the sensor device for transmitting data from the sensor device to a remote location, while the receiver is operatively connected to the control device for inducing an automatic actuation thereof in response to control signals received from the remote location via the signal receiver.

The control exercised from the remote location may be effectuated with respect to the sensor device itself. In that case, the control device is operatively connected to the sensor device for enabling remote operation thereof. Alternatively or additionally, the control device may be connected to another medical instrument such as a surgical implement. The control device may be a robot mechanism for shifting the surgical implement or sensor device relative to the patient in response to the control signals. Thus, a physician at one location is empowered to operate diagnostic and treatment instrumentalities at another location, wherever the patient is located.

The present invention thus facilitates the making of house calls, not only by a physician with a medical bag, but by a hospital operating room. Where general surgery is to be performed, it is recommended that the mobile operating room include trained personnel such as an anaesthesiologist. This expert may be necessary to ensure the proper administration of drugs, oxygen, saline solution, and other customary fluids to the patient. However, it is possible for the administration of these necessities to be carried out partially by robotic mechanisms under the control of a supervising physician at a remote location.

In accordance with another feature of the present invention, motion detectors are disposed in the cargo compartment or mobile operating room. The motion detectors track the motion of a patient relative to the operating equipment. A motion compensation controller is operatively connected to the robot mechanism(s) for modifying the actuation thereof in accordance with detected motion.

It is contemplated that the signal receiver and the transmitter are a wireless receiver and a wireless transmitter, respectively. This enables the performance of diagnostic and therapeutic medical procedures regardless of the location of the patient. Of course, if the patient is found near a telecommunications terminal connected to a telephone line, a DSL. line, a coaxial or optical cable, or other physical telecommunications link, then it is possible to establish a wired communications pathway between the mobile medical facility and the physician.

As discussed in detail hereinafter, the sensor device may include a scanner. The scanner may be an optical camera, an infrared camera, an ultrasonic scanner or based on another modality (magnetic resonance, X-rays, etc.).

A related medical method comprises, in accordance with the present invention, physically moving a medical facility including an enclosed space or chamber from a first location to a second location, introducing a patient into the enclosed space at the second location, thereafter receiving a signal from a remote location, and in response to control information in the signal, automatically operating at least one medical device in the enclosed space to perform a medical procedure on the patient disposed in the enclosed space. Thus, the operating of the medical device is undertaken without human intervention in the mobile medical facility. The device is operated under direct control of the signal transmitted from a physician at the remote location. This is telemedicine effectuated on a patient in a mobile medical facility.

It is contemplated that the medical facility is provided with a propulsion apparatus or engine, so that the moving of the medical facility is undertaken in part by activating the propulsion apparatus. More specifically, the medical facility may be on a truck or other wheeled land vehicle so that the moving of the medical facility includes rolling of the wheels. Alternatively, the mobile medical facility may be provided on an aircraft (helicopter, airplane, balloon, spacecraft, etc.) or water-going craft (ship, barge, submarine, etc.).

Again, it is contemplated that the medical facility, including the enclosed space or chamber, is stationary during the performance of the medical procedure. However, it is possible to conduct an examination or treatment even when the medical facility is in motion. In that case, the medical facility may include sensors for tracking the motion of the patient and/or the medical device and means for compensating the relative motion of the patient and the medical device induced by movement of the medical facility under action of the propulsion mechanism.

Where the medical procedure is a surgical procedure and the medical device includes a medical instrument and a robot mechanism, the operating of the medical device includes actuating the robot mechanism to move the medical instrument relative to the patient.

In a particularly versatile embodiment of the invention, the control signal or signals are received via a wireless signal receiver and a wireless telecommunications link such as a microwave or satellite transmission pathway.

In accordance with another feature of the present invention, a scanner disposed in the enclosed space of the mobile medical facility is operated to garnish images of the patient. The images are transmitted from the medical facility to the remote location for providing, to a remotely located physician or surgeon, real-time information pertaining to the patient. Optimally, the scanner is activated under control from the remote location. Thus, the remote physician or surgeon is able to select the images needed by the physician or surgeon to perform the medical procedure required on the patient.

Accordingly, a particular embodiment of a medical treatment system in accordance with the present invention comprises a self-propelled vehicle having a cargo compartment, an operating table disposed in the cargo compartment for supporting a patient, and at least one robotic surgical mechanism disposed in the cargo compartment for surgically operating on the patient supported by the table. A signal receiver disposed on the vehicle is operatively connected to the robotic surgical mechanism for inducing an automatic actuation thereof in response to control signals received from a remote location via the signal receiver.

A scanner disposed in the cargo compartment and connected to a transmitter for sending images from the cargo compartment to the remote location may include a camera mounted to a movable support such as an endoscopic (laparoscopic, arthroscopic, thoracoscopic, colonoscopic, etc.) instrument. A shifting mechanism may be operatively connected to the support and to the signal receiver for moving the support and aiming the camera in response to signals from the remote location.

A related medical treatment method comprises, in accordance with a specific embodiment of the present invention, providing a surgical operating room containing an operating table, physically moving the operating room from a first location to a second location, disposing a patient on the operating table at the second location, thereafter receiving a signal from a remote location, and automatically operating at least one robotic surgical mechanism in the operating room, in response to control information in the signal, to perform a surgical procedure on the patient disposed on the operating table.

In a telemedicine system including a mobile medical facility as described hereinabove, at least some information transmitted between a patient's location in a medical facility vehicle, on the one hand, and a physician's location, on the other hand, occurs essentially in parallel and simultaneously along two separate transmission paths, thus effectuating a redundancy for safety assurance purposes. The duplicated transmission may be of a control signal from the physician or, alternatively, feedback from the patient's location providing essentially real-time input to the physician of the patient's condition.

Thus, remote surgery as described herein is advantageously implemented with at least two fully redundant communications pathways between manipulator and instrument terminals and with an automatic “hot-swapping” between the pathways and entails having on hand standby terminal units. As with all backup operations, every other factor being equal, a redundancy is maximized when a physical separation between redundant functionality is maximized. In a context of telesurgery, physical separation of pathways means ideally that communications paths are physically separate; for example, if one path includes the public telephone network and a telephone line, a back up path might be via satellite or microwave communications and a satellite dish. Sections of the public telephone network itself, particularly a long-distance network, might be proven by a supplier to have sufficient redundancy built in to themselves constitute acceptably reliable links according to a sense of redundancy in a telesurgical system.

The present invention provides an improved method or apparatus for performing remotely executed medical procedures including, but not limited to, surgical operations. In such remotely executed medical procedures, one or more medical personnel (e.g., physicians) are beyond a range of direct manual contact with the patient.

In a method and/or apparatus in accordance with the present invention, the control exercisable by the operating physician is enhanced. The method and/or apparatus is particularly usable with minimally invasive medical procedures such as those carried out with endoscopic and/or laparoscopic equipment.

The present invention provides a method or apparatus which facilitates the performance of operations by surgeons from all over the world.

### BRIEF DESCRIPTION OF THE DRAWINGS

FIG. 1 is a schematic perspective or isometric view, partially broken away, of a mobile medical facility in accordance with the present invention.

FIG. 2 is partially a cross-sectional view, partially a side elevational view and partially a block diagram of the facility of FIG. 1.

FIG. 3 is a block diagram of a computer shown in FIG. 2, showing selected functional components of the computer and connections thereof with other functional blocks shown in FIG. 2.

FIG. 4 is a block diagram of a remotely controlled medical system with redundant transmission paths in accordance with the present invention.

FIG. 5 is a block diagram illustrating selected details of the system of FIG. 4.

FIG. 6 is a block diagram illustrating additional selected details of the system of FIG. 4.

### DETAILED DESCRIPTION OF THE DRAWINGS AND DESCRIPTION OF THE PREFERRED EMBODIMENTS

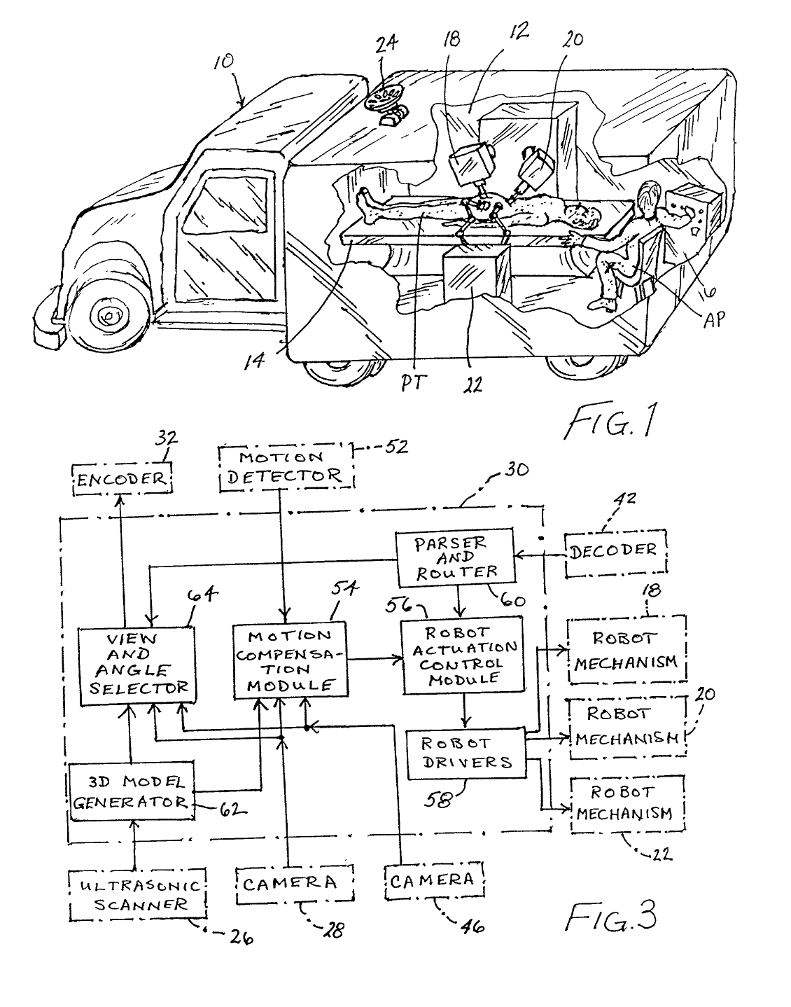

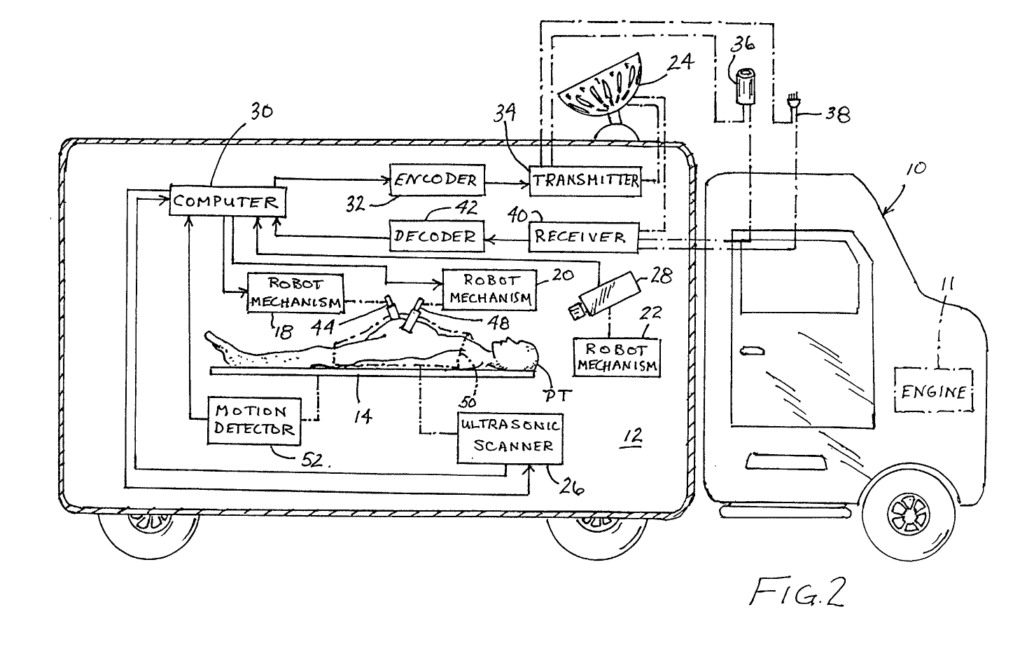

As illustrated in FIG. 1, a mobile medical facility movable to different locations, in particular to the location of a patient includes a vehicle 10 such as a truck propelled by an engine 11 (FIG. 2). Vehicle 10 generally remains stationary during the performance of a medical procedure inside a cargo compartment or medical treatment chamber 12 of the mobile medical facility. Compartment or chamber 12 is an enclosed space provided with a platform or table 14 on which a patient PT may be disposed for the duration of a medical diagnostic and/or treatment procedure. It is contemplated that an attending physician AP such as an anaesthesiologist or other medical case personnel is present in compartment or chamber 12 during the medical procedure. The anaesthesiologist AP has access to conventional equipment 16 for sedating, oxygenating and monitoring the patient PT. However, at least several steps in the medical procedure, including the shifting of medical instruments relative to the patient PT, are carried out automatically by robot mechanisms 18, 20 , 22 under control of a physician at a remote location outside of the vehicle 10. The remote physician may be located in a different part of the same city, town, municipality or other geopolitical unit, or even on a different continent. A wireless antenna 24 such as a satellite dish is provided for enabling wireless transmission of control signals from the remote physician and the transmission of feedback regarding the patient PT to the remote physician.

Many of the elements of the system of FIGS. 1 and 2, including an electromagnetic signaling link, are described in a prior art telesurgery or telemedicine system described in U.S. Pat. Nos. 5,217,003, 5,217,453, or 5,368,015. Those patents are hereby incorporated by reference.

As illustrated in further detail in FIG. 2, an ultrasonic scanner 26 and a camera 28 are provided in compartment or chamber 12 for collecting information on patient PT. Other kinds of sensor devices (none shown) may be provided in compartment or chamber 12 for collecting data on other parameters indicative of the patient's condition. Such sensors include temperature sensors, blood oxygenation detectors, heart rate sensors, blood pressure measurement devices, respiration monitors, etc. The data is collated by a computer 30 and transmitted to the remote physician via an encoder 32 (optionally with encryption capability) and a transmitter 34. Transmitter 34 is operatively connected to a telecommunications link including satellite dish 24, a coaxial or optical cable connection 36, or telephone (twisted pair) network line 38. The same telecommunications link or a different link is connected on an incoming side to a signal receiver 40 and a decoder 42. Decoder 42 is connected to computer 30.

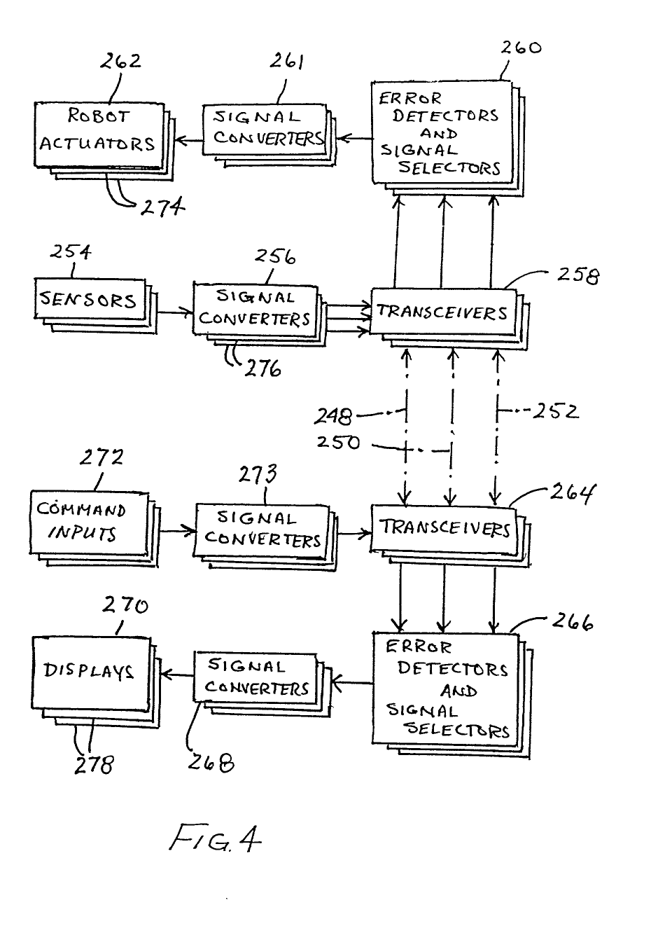

In response to control signals arriving from the remote physician via satellite dish 24, cable 36 or telephone line 38, computer 30 actuates robot mechanisms 18, 20, and 22. These robot mechanisms 18, 20, and 22 determine the positioning and operation of medical diagnostic and/or treatment devices such as endoscopic or laparoscopic instruments 44, 48 and cameras 28, 46 (FIG. 3). Cameras 28 and 46 are operatively connected to computer 30 for providing image data thereto. One or more cameras, e.g., camera 46, may be disposed on an endoscopic or laparoscopic instrument 44, 48 for obtaining images from inside the patient PT. It is contemplated that the image data generated by cameras 28 and 46 is relayed to the remote physician via encoder 32 and transmitter 34.

Also in response to control signals arriving from the remote physician via satellite dish 24, cable 36 or telephone line 38, computer 30 may alter the operation of ultrasonic scanner 26, for instance, by varying the frequencies of ultrasonic energizing pulses. Scanner 26 is connected to a transducer carrier 50 which is placed in operative contact with the patient PT. Scanner 26 and carrier 42 may take a form described and illustrated in U.S. Pat. Nos. 5,871,446, 6,023,632, or 6,106,463, the disclosures of which are hereby incorporated by reference.

As further illustrated in FIG. 2, at least one motion detector 52 is provided in compartment or chamber 12. Motion detector 52 is operatively coupled to operating table 14 for measuring movements of that object. Motion detector 52 thus provides computer 30 with position feedback when a medical procedure is undertaken during motion of vehicle 10.

In addition to monitoring the instantaneous position (including orientation) of table 14, computer 30 optimally monitors the position of patient PT and of various internal organs of the patient. In actuating robot mechanisms 18, 20, 22 during a medical procedure, computer 30 is thereby able to automatically correct for displacements of the patient owing to the motion of the operating table occasioned by the motion of vehicle 10. To that end, as illustrated in FIG. 3, computer 30 includes a motion compensation module 54 operatively connected at inputs to motion detector 52 and cameras 28, 46 and at an output to a robot actuation control module 56. Control module 56 is operatively tied to robot mechanisms 18, 20, 22 via robot drivers 58 and controls the directions, rates, and degrees of operation of the robot mechanisms in response to control signals received from a remote physician via receiver 40 (FIG. 2), decoder 42 and a parser and router component 60 of computer 30. Control of robot mechanisms 18, 20, 22 is fine tuned by module 56 in response to signals from motion compensation module 54. Motion compensation module 54 and robot control module 56 also cofunction to facilitate or enable the performance of medical procedures on naturally moving organs of a patient such as a heart or eye. Apparatus for tracking and compensating for natural patient motion is well known in the fields of computer assisted cardiac surgery and laser eye surgery.

As further illustrated in FIG. 3, computer 30 includes a 3D model generator 62 operatively connected to ultrasonic scanner 26 and, optionally, cameras 28 and 46, for assembling three-dimensional models of a patient's internal organic structures from ultrasonically and optically gathered data. Three-dimensional model generation is disclosed in U.S. Pat. Nos. 5,871,446, 6,023,632, and 6,106,463. As further disclosed in those patents, a view and angle selector 64 may be provided for enabling a physician to choose an angle, a cross-sectional slice, and a degree of magnification of an image to view during an operation. View and angle selector 64 is operatively connected at inputs to 3D model generator 62 and cameras 28, 46 and at an output to encode 32 for providing the remote physician with an image selected among those captured by cameras 28 and 36 and constructed from a three-dimensional model derived by generator 62. View and angle selector 64 performs a selection in response to instructions received from the remote physician via receiver 40, decoder 42, and parser and router 60.

It is to be noted that where a broadband, high-data communications link is available, some or all of the functions of view and angle selector 64 may be performed at the remote station. In that case, the video images from cameras 28 and 46, as well as the three-dimensional models from generator 62, may be transmitted by encoder 32 and transmitter 34 to the remote station.

The various components of computer 30 may be realized as generic digital processing circuits modified by programming to accomplish the intended functions. However, it is also possible for one or more functions, such as image processing, to be implemented by dedicated circuits, as stand alone units or as plug-in boards on a frame of computer 30.

Computer 30 generally receives input from other patient conditions sensors including temperature sensors, blood oxygenation detectors, heart rate sensors, blood pressure measurement devices, respiration monitors, etc. (none shown). Computer 30 may process this information prior to transmission thereof to the remote station.

Cameras 28, 46 may be optical or infrared devices. Other scanners may be provided, such as a magnetic resonance imaging apparatus, an X-ray device (e.g. a CAT scan) etc. Output from these scanners may be processed by computer 30 and transmitted to the remote physician.

In use, vehicle 10 of the mobile medical facility is moved from one location to another, where a patient is introduced into compartment chamber 12. That second location may be at a medical facility or virtually anywhere accessible by vehicle 10. Where a telecommunications network access port is available, such as an optical coupling, a cable link-up, a DSL connection, a telephone socket, etc., a connection is made thereto, for instance, via coaxial or optical cable connection 36 or telephone (twisted pair) network line 38. Thereafter, a signal arriving from a physician at a remote location is isolated by receiver 40 and adapted by decoder 42. Control information in the incoming signal is detected and interpreted by parser and router 60 and robot actuation control module 56. In response to the control information, module 56 automatically operates at least one medical device in compartment or chamber 12 to perform a medical procedure on the patient PT. The medical device may be a diagnostic device such a camera 28 or scanner 26 or a treatment device such as an endoscopic or laparoscopic surgical instrument 44, 48.

It is contemplated that vehicle 10, including enclosed space or chamber 12, is stationary during the performance of a medical procedure. However, it is possible to conduct an examination or treatment in chamber 12 even when vehicle 10 is in motion. As discussed above, motion compensation module 54 tracks the motion of the patient PT and/or the medical devices 26, 28, 44, 46, 48 being controlled by the remote physician and compensates for the relative motion of the patient PT and the medical device 26, 28, 44, 46, 48 induced by movement of vehicle 10 under action of the propulsion mechanism or engine 11.

The medical systems discussed above with reference to FIGS. 1-3 may be implemented with a plurality of redundant signal transmission paths or communications pathways 248, 250 and 252, as shown in the generic system of FIG. 4. Sensors 254 such as various scanners are disposed in a mobile medical facility or vehicle for generating various signals indicative of the condition of a patient located in the vehicle. Generally, sensors 254 include analog, as well as possible digital, sensors. Signal converters 256 are provided for changing the format of the sensor signals, if required for transmission over communication pathways 248, 250, 252. The change in format may be from analog to digital, to encrypted, to amplitude or frequency modulated, etc. The converted signals are supplied to a plurality of transceiver units 258 equal in number to transmission paths 248, 250, 252. Via transmission paths 248, 250, 252 transceivers 258 receive control or command signals from a physician or other specialist at a remote location. The incoming control or command signals are examined by error detectors and signal selectors 260 in the mobile medical facility or vehicle to detect and select a control or command signal which has been accurately conveyed over a transmission path 248, 250, 252. If necessary, the selected signal is transformed from a digital signal to an analog signal by a converter 261 and fed to a respective robot actuator or servomechanism 262 for moving a medical diagnostic or therapeutic instrument relative to the patient under the control of the remotely located physician or medical specialist.

The signals generated by sensors 254 are transmitted in appropriate format (digital, analog, modulated, compressed, encrypted, etc.) from the mobile medical facility or vehicle over transmission paths 248, 250, 252 to transceivers 264 at the location of the remote physician or specialist. Transceivers 264 furnish the incoming sensor signals to an error detection and signal selection module 266 which analyzes the sensor signals for discrepancies and selects a signal having little or no discernible error. That selected signal is transformed, if necessary, by a signal converter 268 and provided to an appropriate display or output peripheral 270. Various command or control peripherals or input units 272 are provided at the physician location for collecting instructions or commands for relay to the patient location via redundant transmission paths 248, 250, 252. Signal converters 273 are provided, if necessary, for transforming control and command signals from command inputs 272 into a format appropriate for conveyance over the respective transmission paths 248, 250, 252.

The main redundancy provided in the system of FIG. 4 is the multiple pathways 248, 250, and 252 for signal transmission. This redundancy is particularly important where the physician is located at an extreme distance from the patient. Where the physician is located in the same room or on the same premises, the redundancy is not necessarily as vital.

As illustrated in FIG. 4, various hardware elements may be provided with backup components, for instance, backup actuators 274, backup signal converters 276, and backup displays 278. Generally, these backup components are dormant unless a failure or malfunction is detected in the primary unit. In that event, a switchover is effectuated to a backup component. However, where hardware elements are required for multiple signal transmission in parallel over transmission paths 248, 250, and 252, those hardware elements function simultaneously, in parallel and independently. Of course, one or more of these parallel functioning components may be provided with a respective backup.

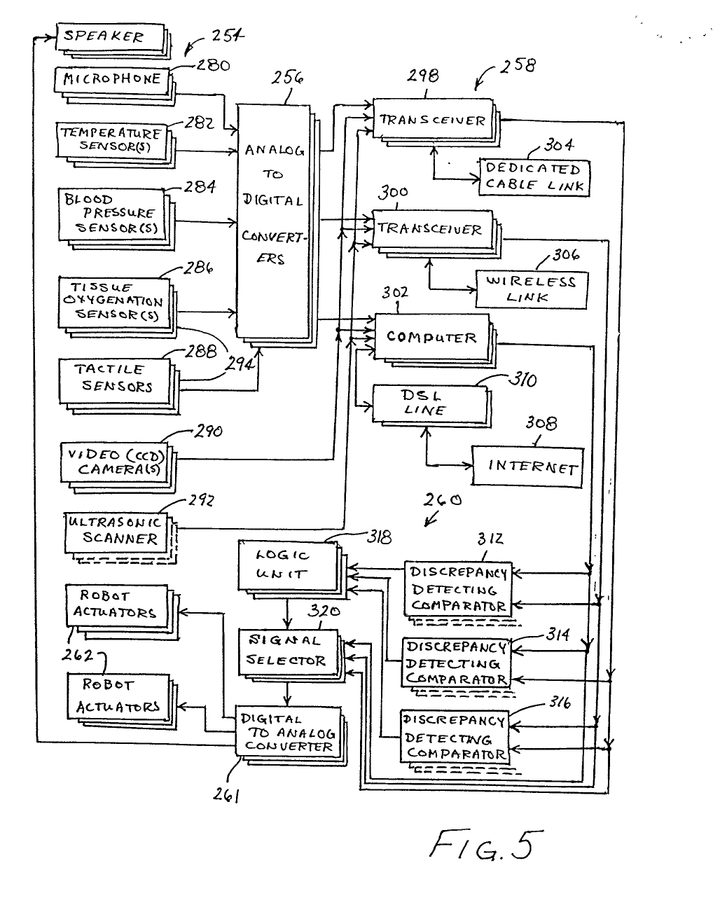

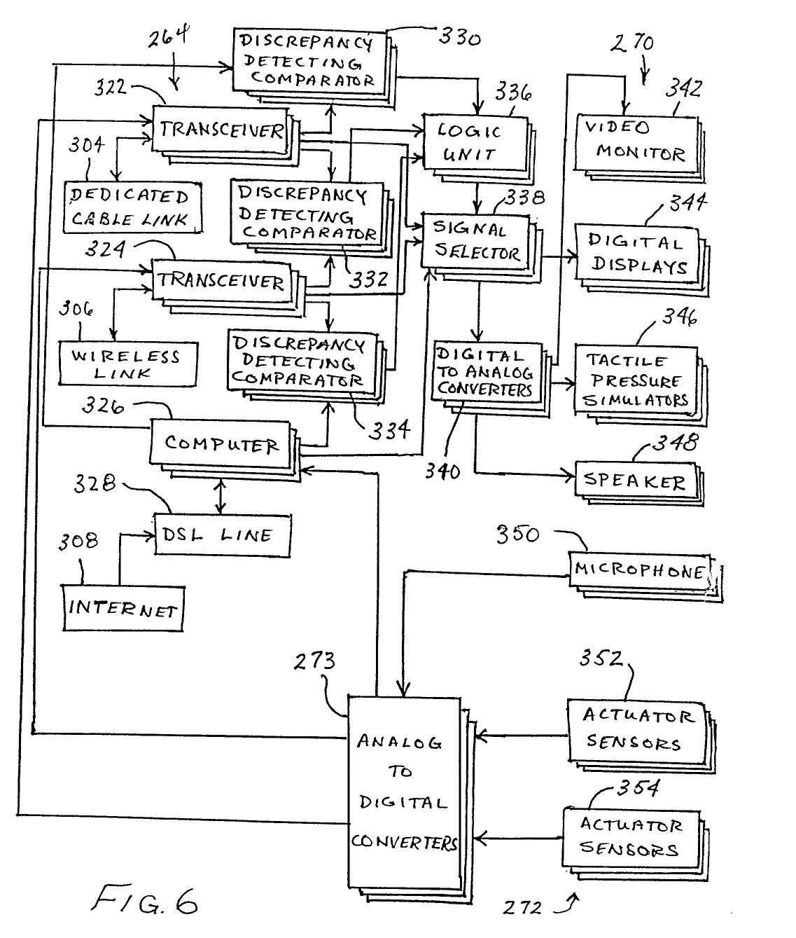

FIG. 5 shows, in greater detail, components of the generic system of FIG. 4 which are disposed in a mobile medical facility or vehicle. Sensors 254 of the system of FIG. 4 may include a microphone 280, one or more temperature sensors 282, one or more blood pressure sensors 284, one or more tissue oxygenation sensors 286, one or more tactile sensors 288, a video camera 290 such as a CCD or CMOS APS device, and an ultrasonic scanner 292. As discussed above, these sensors may be provided with one or more backups, generically 294, which lie dormant except in the event of a malfunction of the primary unit. Microphone 280, temperature sensors 282, blood pressure sensors 284, tissue oxygenation sensors 286, and tactile sensors 288 generally produce analog output signals. These analog sensor signals are fed to converters 256 which transform the sensor signals into suitable format for conveyance over transmission paths or communications pathways 248, 250, 252 (FIG. 4). As discussed above, the transformation of the sensor signals by converters 256 may include analog to digital conversion, encryption, amplitude or frequency modulation, etc. Transceiver units 258 include transceivers 298 and 300 and a computer 302. In the detailed example of FIGS. 5 and 6, transmission paths 248, 250, and 252 are implemented respectively by a dedicated wire or optical-fiber cable link 304, a wireless (e.g., satellite) link 306, and a global computer network (the Internet) 308. Computer 302 is coupled to the Internet 308 via a suitable broadband connection such as a DSL line 310.

As further illustrated in FIG. 5, error detection and signal selection module 260 includes a plurality of discrepancy detecting comparators 312,314, and 316 which analyze control and command signals arriving from a physician location over respective pairs of transmission links 248, 250, 252. To that end, comparator 312 is connected at a respective pair of inputs to transceiver 300 and computer 302, while comparator 314 has two inputs coupled to transceiver 298 and computer 302, comparator 316 being tied at inputs to transceivers 298 and 300. Error detection and signal selection module 260 further includes a logic unit 318 receiving the results of the discrepancy checking by comparators 312, 314, and 316. Logic unit 318 determines which of the incoming control and command signals arriving via transceivers 298 and 300 and computer 302 has little or no discernible error. That signal is selected for use and is passed through by a signal selector 320 under the control of logic unit 318. The selected signal is transformed, if necessary, by signal converter 261 and delivered to a respective robot actuator 262.

As illustrated in FIG. 6, transceivers 264 include a pair of transceivers 322 and 324 connected to dedicated cable link 304 and wireless link 306, respectively, and further include a computer 326 connected to the Internet 308 via a DSL line 328 or other broadband high-speed connection. Error detection and signal selection unit 266 includes a plurality of discrepancy detecting comparators 330, 332, and 334 operatively tied at their inputs to respective pairs of transceivers 322 and 324 and computer 326. Comparators 330, 332, 334 examine sensor signals arriving from a mobile medical facility or vehicle over respective pairs of transmission links 248, 250, 252 (FIG. 4), to detect discrepancies or errors in the transmitted signals. Error detection and signal selection unit 266 further includes a logic unit 336 receiving the results of the discrepancy checking by comparators 330, 332, and 334. Logic unit 336 determines which of the incoming control and command signals arriving via transceivers 322 and 324 and computer 326 has little or no discernible error. That signal is selected for use and is passed through by a signal selector 338 under the control of logic unit 336. The selected signal is transformed, if necessary, by signal converter 340 and delivered to a respective displays 278, including a video monitor 342, a bank of digital displays 344 and other indicators and communication peripherals including tactile pressure simulators 346 and a speaker 348. Tactile pressure simulators 346 provide a physician with a sense of touch, including in particular pressure sensations, derived from input provided by tactile sensors 288 (FIG. 5).

Command inputs 272 include a microphone 350 and actuator sensors 352 and 354 representing the input from different manually operated controls, including, for instance, buttons, levers, slidable switches, joysticks, and other input devices enabling remote control of a medical instrument which is, for example, pivotable about a plurality of rotation axes and translatable along three coordinate axes, and which has one or more operable appendages including jaws (scissors, graspers, forceps, clamps), cauterization elements, injectors, etc. Microphone 350 and sensors 352 and 354 are connected to signal converters 273 which transform voice and control signals into suitable format for conveyance over transmission paths or communications pathways 248, 250, 252 (FIG. 4). As discussed above, the transformation of the control and command signals by converters 273 may include analog to digital conversion, encryption, amplitude or frequency modulation, etc.

In the mobile medical system of FIGS. 1-3 with redundancies as discussed with hereinabove with reference to FIGS. 4-6, a video signal is generated by cameras 290 and/or ultrasonic scanner 292 in the mobile medical facility or vehicle and encodes an image of the patient. The video signal is transmitted essentially simultaneously as a first, second, and third signal over transmission paths or communications pathways 248, 250, and 252 to the physician location. At that receiving location, the image of the patient is produced on the image-reproduction video monitor 342 from at least one of the first, second, and third signals. (Only two signals may be sufficient in many cases.) In response to the image production, an operator (physician) manipulates input devices, that manipulation being detected and encoded by actuator sensors 352 and/or 354 in a control or command signal which is transmitted essentially simultaneously as a fourth, fifth, and sixth signal over parallel transmission paths or communications pathways 248, 250, 252 to the mobile medical facility or vehicle. A medical instrument in the vehicle is automatically moved to conduct a medical operation on the patient in response to at least one of the fourth, fifth and sixth signals.

The checking for signal discrepancies by comparators 312, 314, 316 and 330, 332, 334 may entail a review of parity bits to determine the existence of a parity error indicative of data loss. Other types of comparison techniques include periodic sampling of the respective digital streams (in the case of digital signal transmission) and comparing of corresponding data bits or bytes.

Ultrasonic scanner 292 may take a form as disclosed in U.S. Pat. Nos. 5,871,446, 6,023,632, or 6,106,463. Alternative sources of image data include infrared sensors, CAT scanners and MRI machines. The audio signals in the one direction may encode sounds made by the patient or by attending personnel in the area of the patient, while audio signals from the remote physician may include verbal instructions to the patient and/or to the attending personnel.

Although the invention has been described in terms of particular embodiments and applications, one of ordinary skill in the art, in light of this teaching, can generate additional embodiments and modifications without departing from the spirit of or exceeding the scope of the claimed invention. For instance, it is to be noted that the mobile medical facility may be provided on an aircraft (helicopter, airplane, balloon, spacecraft, etc.), a train, or water-going craft (ship, barge, submarine, etc.). It is to be noted, in addition, that in some circumstances, the physician in charge may be located in compartment or chamber 12 with the patient PT but so remote from the patient that direct manual operation of diagnostic and therapeutic instruments by the physician is not possible. Accordingly, it is to be understood that the drawings and descriptions herein are proffered by way of example to facilitate comprehension of the invention and should not be construed to limit the scope thereof.

What is claimed is:

1. A medical treatment method comprising:
providing a surgical operating room containing an operating table;
physically moving said operating room from a first location to a second location;
at said second location, disposing a patient on said operating table;
after the disposing of said patient on said operating table, generating, at said second location, a video signal encoding an image of said patient disposed on said operating table;
transmitting said video signal as a first signal over a first communications path to a third location beyond a range of direct manual contact with the patient;
substantially simultaneously transmitting said video signal as a second signal to said third location over a second communications path different from said first communications path;
producing said image on an image reproduction device at said third location from at least one of said first signal and said second signal;
in response to said image production, generating a control signal at said third location;
transmitting said control signal as a third signal over a third communications path to said second location;
substantially simultaneously with the transmitting of said third signal, transmitting said control signal as a fourth signal to said second location over a fourth communications path different from said third communications path; and
in response to at least one of said third signal and said fourth signal, automatically operating at least one robotic surgical mechanism in said operating room to perform a surgical procedure on said patient disposed on said operating table.

2. The method defined in claim 1 wherein said first communications path and said third communications path are enabled by a common set of hardware or instrumentalities, said second and said fourth communications path being enabled by a common set of hardware or instrumentalities.

3. The method defined in claim 1 wherein the generating of said video image includes operating an endoscopic instrument inserted into the patient to direct an imaging device on said endoscopic instrument towards an area of interest inside the patient.

4. The method defined in claim 1 wherein said communications paths all include electromagnetic signaling links, the transmitting of said first signal, said second signal, said third signal, and said fourth signal including the generating of digital electrical signals.

5. The method defined in claim 1 wherein said medical instrument includes a surgical tool, the actuating of said medical instrument including moving said tool relative to the patient.

6. The method defined in claim 1 wherein said communications paths include wireless telecommunications links.

7. The method defined in claim 1 wherein said operating room is provided with a propulsion apparatus, the moving of said operating room including the activating of said propulsion apparatus.

8. A medical treatment system comprising:
a self-propelled vehicle having a cargo compartment;
an operating table disposed in said cargo compartment for supporting a patient;
at least one robotic surgical mechanism disposed in said cargo compartment for surgically operating on the patient supported by said table; and
a signal receiver disposed on said vehicle, said signal receiver being operatively connected to said robotic surgical mechanism for inducing an automatic actuation thereof in response to control signals received from a remote location via said signal receiver, said signal receiver being connected to at least two communications pathways for receiving redundant copies of said control signals.

9. The system defined in claim 8, further comprising:
a scanner disposed in said cargo compartment; and
a transmitter disposed on said vehicle and operatively connected to said scanner for transmitting images from said cargo compartment to said remote location.

10. The system defined in claim 9, further comprising a support and a shifting mechanism operatively connected to said support for moving said support, said scanner being mounted to said support.

11. The system defined in claim 10 wherein said support is an endoscopic instrument insertable into the patient on said operating table.

12. The system defined in claim 10 wherein said shifting mechanism is operatively connected to said signal receiver for aiming said scanner under control from said remote location.

13. The system defined in claim 9, further comprising:
motion detectors disposed in said cargo compartment; and
a motion compensation controller operatively connected to said robotic surgical mechanism for modifying the actuation thereof in accordance with detected motion.

14. The system defined in claim 13 wherein said motion detector is operatively connected to said scanner for analyzing images therefrom to quantitatively determine motion of at least a portion of the patient.

15. The system defined in claim 9 wherein said receiver includes a wireless receiver and said transmitter includes a wireless transmitter.

16. The system defined in claim 9 wherein said transmitter connected to at least two communications pathways for transmitting redundant copies of said images.

17. The system defined in claim 8, further comprising a computer operatively connected to said signal receiver and said robotic surgical mechanism for controlling operation of same in response to instructions received from said remote location via said signal receiver.

18. The system defined in claim 8 wherein said receiver includes a wireless receiver.

19. The system defined in claim 8, further comprising means operatively connected to said receiver for selecting one of said redundant copies of said control signal.

20. A medical treatment method comprising:
providing a surgical operating room containing an operating table;
physically moving said operating room from a first location to a second location;
at said second location, disposing a patient on said operating table;
after the disposing of said patient on said operating table, receiving a signal from a remote location; and
in response to control information in said signal, automatically operating at least one robotic surgical mechanism in said operating room to perform a surgical procedure on said patient disposed on said operating table.

21. The method defined in claim 20, further comprising generating a video image of the patient and transmitting said video image to said remote location redundantly along two separate communications pathways.

22. The method defined in claim 21 wherein the generating of said video image includes operating an endoscopic instrument inserted into the patient to direct an imaging device on said endoscopic instrument towards an area of interest inside the patient.

23. The method defined in claim 20 wherein said signal is a control signal selected from two redundant signals transmitted from said remote location over two different and separate communications pathways, the receiving of said signal including receiving said redundant signals over said communications pathways, the receiving of said signal further including selecting one of said redundant signals as said control signal.

24. The method defined in claim 23 wherein said communications pathways include wireless telecommunications links.

25. The method defined in claim 23 wherein said communication pathways include electromagnetic signaling links, said redundant signals being digital electrical signals.

26. The method defined in claim 23 wherein the selecting of said one of said redundant signals occurs automatically in accordance with preselected signal evaluation criteria.

27. The method defined in claim 20 wherein said medical instrument includes a surgical tool, the actuating of said medical instrument including moving said tool relative to the patient.

28. The method defined in claim 20 wherein said operating room is provided with a propulsion apparatus, the moving of said operating room including the activating of said propulsion apparatus.

29. A medical treatment system comprising:
a self-propelled vehicle having a cargo compartment;
an operating table disposed in said cargo compartment for supporting a patient;
at least one robotic surgical mechanism disposed in said cargo compartment for surgically operating on the patient supported by said table; and
a signal receiver disposed on said vehicle, said signal receiver being operatively connected to said robotic surgical mechanism for inducing an automatic actuation thereof in response to control signals received from a remote location via said signal receiver.

30. The system defined in claim 29, further comprising:
a scanner disposed in said cargo compartment; and
a transmitter disposed on said vehicle and operatively connected to said scanner for transmitting images from said cargo compartment to said remote location.

31. The system defined in claim 30 wherein said receiver includes a wireless receiver and said transmitter includes a wireless transmitter.

32. The system defined in claim 30 wherein said transmitter is connected to at least two communications pathways for transmitting redundant copies of said images to said remote location.

33. The system defined in claim 29 wherein said signal receiver is connected to at least two communications pathways for receiving redundant copies of said control signals

34. A medical system comprising:
a self-propelled vehicle having a cargo compartment;
a sensor device disposed in said cargo compartment for collecting information on a patient in said cargo compartment;
at least one control device disposed in said cargo compartment for operating a medical instrument relative to the patient; and
communications components including a signal receiver and a transmitter disposed on said vehicle, said transmitter being operatively connected to said sensor device for transmitting data from said sensor device to a remote location, said receiver being operatively connected to said control device for inducing an automatic actuation thereof in response to control signals received from said remote location via said signal receiver.

35. The system defined in claim 34 wherein said medical instrument is said sensor device, said control device being operatively connected to said sensor device for enabling remote operation thereof.

36. A medical method comprising:
providing a medical facility including an enclosed space;
physically moving said medical facility from a first location to a second location;
introducing a patient into said enclosed space at said second location;
after the introducing of said patient into said enclosed space, receiving a signal from a remote location; and
in response to control information in said signal, automatically operating at least one medical device in said enclosed space to perform a medical procedure on said patient disposed in said enclosed space.

37. The method defined in claim 36 wherein said medical facility is provided with a propulsion apparatus, the moving of said medical facility including the activating of said propulsion apparatus.
